(19) United States
(12) Patent Application Publication
(10) Pub. No.: US 2017/0196797 A1
(43) Pub. Date: Jul. 13, 2017

(54) COMPOSITION CONTAINING GLYCINE GRACILIS OIL

(71) Applicant: AMOREPACIFIC CORPORATION, Seoul (KR)

(72) Inventors: Young Gyu KANG, Yongin-si, Gyeonggi-do (KR); Ok Chan LEE, Yongin-si, Gyeonggi-do (KR); Yong Deog HONG, Yongin-si, Gyeonggi-do (KR); Myeong Hun YEOM, Yongin-si, Gyeonggi-do (KR); Jun Seong PARK, Yongin-si, Gyeonggi-do (KR)

(21) Appl. No.: 15/324,521

(22) PCT Filed: Jun. 29, 2015
(86) PCT No.: PCT/KR2015/006647

§ 371 Date: Jan. 6, 2017

(30) Foreign Application Priority Data
Jul. 16, 2014 (KR) 10-2014-0090008

(51) Int. Cl. A61K 8/92; A61Q 17/00; A61Q 19/08; A61Q 19/02; A61Q 19/00; A61K 36/48

(52) U.S. Cl. CPC A61K 8/922; A61Q 19/00; A61K 36/48; A61Q 19/08; A61Q 19/02; A61Q 17/00

(57) ABSTRACT

Disclosed is a composition containing Glycine gracilis oil as an active ingredient and, particularly, a composition having uses of anti-oxidation, anti-aging, anti-wrinkles, whitening, anti-inflammation, anti-acne, moisturization, atopy alleviation, skin barrier improvement, and the like.

### TECHNICAL FIELD

The present disclosure relates to a composition containing a Glycine gracilis oil as an active ingredient, specifically a composition useful in anti-oxidation, anti-aging, anti-wrinkling, skin whitening, anti-inflammation, anti-acne, skin moisturization, atopy alleviation, skin barrier improvement, etc.

### BACKGROUND ART

Human skin experiences changes with aging due to various intrinsic and extrinsic factors. Intrinsically, the secretion of various hormones regulating metabolism decreases and the function and activity of immune cells decline. As a result, the biosynthesis of immunoproteins and constituent proteins decreases. Extrinsically, as the amount of ultraviolet light reaching the surface of the earth increases due to the destruction of the ozone layer and environmental pollution is aggravated, free radicals, reactive oxygen species, etc. are increased. As a result, various changes occur, including decreased skin thickness, increased wrinkles, decreased skin elasticity, etc.

As aging proceeds, skin experiences change or decrease in the contents and arrangement of the substances constituting the skin, such as collagen, elastin, hyaluronic acid and glycoproteins, and oxidative stresses are caused by free radicals and reactive oxygen species. Also, it is known that aging or UV light increases the biosynthesis of cyclooxygenase-2 (COX-2), which is an enzyme that produces proinflammatory cytokines known to cause inflammations in most cells constituting skin, the biosynthesis of matrix metalloproteinase (MMP), which is an enzyme that degrades skin tissues, and the production of nitric oxide (NO) by inducible nitric oxide synthase (iNOS). That is to say, as the biosynthesis of substrate materials is decreased due to decreased cellular activity and microinflammations with natural intrinsic aging and their degradation is accelerated due to extrinsic factors such as increased stresses caused by various harmful environments and increase in reactive oxygen species, many symptoms of skin aging occur including the destruction and thinning of the skin.

The reactive oxygen species which are produced due to various physical, chemical and environmental factors, including enzymatic reactions, reductive metabolism, chemicals, pollutants, photochemical reactions, etc., are known to cause cellular aging and various diseases including cancer by non-selectively and irreversibly destroying the cellular components such as lipids, proteins, sugars, DNAs, etc. In addition, various peroxides including lipid peroxides produced by lipid peroxidation by these reactive oxygen species cause a variety of diseases by inducing functional disorders through oxidative damage of cells. Therefore, antioxidants such as the compounds that can scavenge the free radicals (free radical scavengers) or the substances that can inhibit the production of peroxides are viewed as candidates for prevention or treatment of aging and various diseases caused by the oxides.

The prior art is described in Korean Patent Publication No. 10-2001-0054290.

### DISCLOSURE

### Technical Problem

In an aspect, the present disclosure is directed to providing a composition containing a Glycine gracilis oil, which is useful in various applications.

### Technical Solution

In an aspect, the present disclosure provides a composition containing a Glycine gracilis oil as an active ingredient.

Specific embodiment 1. A composition for anti-oxidation containing a Glycine gracilis oil as an active ingredient.

Specific embodiment 2. A composition for anti-aging containing a Glycine gracilis oil as an active ingredient.

Specific embodiment 3. The composition according to any of the specific embodiments 1 to 3, wherein the composition delays or improves aging.

Specific embodiment 4. The composition according to any of the specific embodiments 1 to 3, wherein the composition suppresses or inhibits the production of collagenase or suppresses the expression of collagenase.

Specific embodiment 5. The composition according to any of the specific embodiments 1 to 3, wherein the composition improves or alleviates wrinkles.

Specific embodiment 6. A composition for skin whitening, which contains a Glycine gracilis oil as an active ingredient.

Specific embodiment 7. The composition according to the specific embodiment 6, wherein the composition suppresses or inhibits the production of melanin or suppresses the expression of melanin.

Specific embodiment 8. A composition for anti-inflammation containing a Glycine gracilis oil as an active ingredient.

Specific embodiment 9. The composition according to the specific embodiment 8, wherein the inflammation is acne.

Specific embodiment 10. The composition according to the specific embodiment 8 or 9, wherein the composition suppresses or inhibits the production or expression of monocyte chemoattractant protein (MCP).

Specific embodiment 11. A composition for skin moisturization, which contains a Glycine gracilis oil as an active ingredient.

Specific embodiment 12. The composition according to the specific embodiment 11, wherein the composition treats, prevents or improves atopy or psoriasis.

Specific embodiment 13. The composition according to the specific embodiment 11 or 12, wherein the composition strengthens skin barrier.

Specific embodiment 14. The composition according to any of the specific embodiments 1 to 13, wherein the composition contains 0.001-10 wt % of a Glycine gracilis oil.

Specific embodiment 15. The composition according to any of the specific embodiments 1 to 14, wherein the composition is a cosmetic composition.

Specific embodiment 16. The composition according to any of the specific embodiments 1 to 15, wherein the composition is a composition for external application to skin.

Specific embodiment 17. The composition according to any of the specific embodiments 1 to 16, wherein the composition is a pharmaceutical composition.

Specific embodiment 18. The composition according to any of the specific embodiments 1 to 17, wherein the composition is a health food composition.

### Advantageous Effects

A composition according to an exemplary embodiment of the present disclosure, which contains a Glycine gracilis oil as an active ingredient, is effective in anti-aging, anti-wrinkling, skin whitening, anti-inflammation, anti-acne, skin moisturization, atopy alleviation, skin barrier improvement, etc. These effects are better than those of commercially available individual substances known to have respective effects and even than those of other bean oils. In addition, the composition according to an exemplary embodiment of the present disclosure, which contains an oil obtained from a natural plant as an active ingredient, is variously applicable in cosmetic or medical fields because it has few side effects and is hardly likely to cause resistance even when used for a long period of time.

### BRIEF DESCRIPTION OF DRAWINGS

FIG. 1 shows an HPLC analysis result of a Glycine gracilis oil.

FIG. 2 shows an HPLC analysis result of a soybean oil.

FIG. 3 shows an HPLC analysis result of a Glycine gracilis extract.

### BEST MODE

In the present disclosure, “Glycine gracilis” is also called flat bean. The Glycine gracilis of the present disclosure has an oval, flat and longish shape unlike soybean. Glycine gracilis grows as long vines if planted before dog days. But, if planted after dog days, it grows short and has many grains. The leaf of Glycine gracilis is relatively narrow and small and the flower is blue. Glycine gracilis is grown as sprouts because it grows fast and the head part is oval, small, soft and tasty.

In the present disclosure, “Glycine gracilis oil” is not limited as long as it is obtained by a method known in the art. Specifically, it may mean any oil obtained by extracting Glycine gracilis with a hydraulic oil press machine or one obtained by filtering the oil through filter paper, although not being limited thereto.

In an aspect, the present disclosure relates to a composition for anti-oxidation, which contains a Glycine gracilis oil as an active ingredient. In the present disclosure, “anti-oxidation” refers to an effect of delaying, stopping or preventing oxidative processes known in the art without limitation.

In another aspect, the present disclosure relates to a composition for anti-aging, which contains a Glycine gracilis oil as an active ingredient. In the present disclosure, “anti-aging” refers to an effect of delaying, stopping or preventing aging processes known in the art. Specifically, it may mean an effect of effectively suppressing the expression of collagenase, thereby enhancing skin elasticity and alleviating wrinkles by reducing degradation of collagen in skin, although not being limited thereto.

In an aspect of the present disclosure, the composition may delay or improve aging.

In an aspect of the present disclosure, the composition may suppress or inhibit the production of collagenase or suppress the expression of collagenase.

In an aspect of the present disclosure, the composition may improve or alleviate wrinkles.

In another aspect, the present disclosure relates to a composition for skin whitening, which contains a Glycine gracilis oil as an active ingredient. The composition may provide a superior skin whitening effect by suppressing the production of melanin or suppressing the expression of genes involved in the production of melanin.

In another aspect, the present disclosure relates to a composition for anti-inflammation, which contains a Glycine gracilis oil as an active ingredient. In the present disclosure, “anti-inflammation” refers to any kind of activity of suppressing or stopping inflammation. In an aspect of the present disclosure, the inflammation comprises any kind of inflammation in the body including skin. Specifically, the inflammation may be acne, although not being limited thereto.

In an aspect of the present disclosure, the composition may suppress the production of monocyte chemoattractant protein (MCP) or suppress or inhibit the expression of genes involved in its production.

In another aspect, the present disclosure relates to a composition for skin moisturization, which contains a Glycine gracilis oil as an active ingredient. The composition of the present disclosure may enhance the skin barrier function and induce the differentiation of keratinocytes in skin. Accordingly, it may be usefully used to prevent or improve xeroderma, psoriasis, etc. caused by incomplete differentiation of the epidermis.

In this aspect, the composition may treat, prevent or improve atopy or psoriasis. In an aspect, the composition may also strengthen the skin barrier.

In an aspect of the present disclosure, the composition may contain the Glycine gracilis oil in an amount of 0.001 wt % or more, 0.002 wt % or more, 0.003 wt % or more, 0.004 wt % or more, 0.005 wt % or more, 0.006 wt % or more, 0.007 wt % or more, 0.008 wt % or more, 0.009 wt % or more, 0.01 wt % or more, 0.02 wt % or more, 0.03 wt % or more, 0.04 wt % or more, 0.05 wt % or more, 0.06 wt % or more, 0.07 wt % or more, 0.08 wt % or more, 0.09 wt % or more or 0.1 wt % or more and 6 wt % or less, 7 wt % or less, 8 wt % or less, 9 wt % or less, 10 wt % or less, 35 wt % or less, 36 wt % or less, 37 wt % or less, 38 wt % or less, 39 wt % or less or 40 wt % or less, for example, 0.001-10 wt %, 0.001-10 wt %, 0.005-9 wt %, 0.01-8 wt %, 0.05-7 wt % or 0.1-6 wt %. When the amount of the Glycine gracilis oil is smaller than the above-described range, the above-described effects may be insignificant. And, when the amount is larger than the above-described range, the contents of other ingredients may be affected without distinct increase in the effects.

In an aspect of the present disclosure, the composition may be a cosmetic composition.

When the cosmetic composition according to the present disclosure is formulated as a cosmetic, it may be prepared into a softening lotion, an astringent lotion, a nourishing lotion, an eye cream, a nourishing cream, a massage cream, a cleansing cream, a cleansing foam, a cleansing water, a powder, an essence, a pack, etc., although not being particularly limited thereto. The composition according to the present disclosure may contain adjuvants commonly used in cosmetic or dermatological fields, such as a fat, an organic solvent, a solubilizer, a thickener, a gelling agent, a softener, an antioxidant, a suspending agent, a stabilizer, a foaming agent, an aromatic, a surfactant, water, an ionic or non-ionic emulsifier, a filler, a sequestrant, a chelating gent, a preservative, a vitamin, a blocking agent, a wetting agent, an essential oil, a dye, a pigment, a hydrophilic or lipophilic activator, a lipid vesicle or other ingredients commonly used in cosmetics. The adjuvant is comprised in an amount commonly used in cosmetic or dermatological fields. The composition of the present disclosure may further contain a substance promoting absorption into skin in order to increase the effect of improving skin.

In an aspect of the present disclosure, the composition may be a composition for external application to skin.

The composition of the present disclosure may be a composition for external application to skin. More specifically, it may be used as a composition for external application to skin for anti-oxidation. It may provide a superior antioxidative effect by suppressing the oxidation of DPPH which is an organic radical. Also, the composition of the present disclosure may be used as a composition for external application to skin for anti-aging. It exhibits an excellent anti-aging effect of enhancing skin elasticity and improving wrinkles by effectively suppressing the expression of collagenase in skin and thereby reducing the degradation of collagen in skin. In addition, the composition of the present disclosure may be used as a composition for external application to skin for skin whitening. It may provide a superior skin whitening effect by suppressing the production of melanin. Moreover, the composition of the present disclosure may be used as a composition for external application to skin for skin moisturization. It may strengthen the skin barrier function and induce the differentiation of keratinocytes in skin. Accordingly, it may be usefully used to prevent or improve xeroderma, psoriasis, etc. caused by incomplete differentiation of the epidermis.

When the composition for external application to skin according to the present disclosure is formulated as a cosmetic, it may be prepared into a softening lotion, an astringent lotion, a nourishing lotion, an eye cream, a nourishing cream, a massage cream, a cleansing cream, a cleansing foam, a cleansing water, a powder, an essence, a pack, etc., although not being particularly limited thereto. The composition according to the present disclosure may contain adjuvants commonly used in cosmetic or dermatological fields, such as a fat, an organic solvent, a solubilizer, a thickener, a gelling agent, a softener, an antioxidant, a suspending agent, a stabilizer, a foaming agent, an aromatic, a surfactant, water, an ionic or non-ionic emulsifier, a filler, a sequestrant, a chelating gent, a preservative, a vitamin, a blocking agent, a wetting agent, an essential oil, a dye, a pigment, a hydrophilic or lipophilic activator, a lipid vesicle or other ingredients commonly used in cosmetics. The adjuvant is comprised in an amount commonly used in cosmetic or dermatological fields. The composition of the present disclosure may further contain a substance promoting absorption into skin in order to increase the effect of improving skin.

In an aspect of the present disclosure, the composition may be a pharmaceutical composition.

When the composition according to the present disclosure is used as a pharmaceutical composition, the composition may be prepared into a solid, semi-solid or liquid formulation for oral or parenteral administration by adding a commonly used organic or inorganic carrier.

Formulations for oral administration may comprise a tablet, a pill, a granule, a hard/soft capsule, a dust, a fine granule, a powder, an emulsion, a syrup, a pellet, etc. And, formulations for parenteral administration may comprise an injection, a medicinal drop, an ointment, a lotion, a spray, a suspension, an emulsion, a suppository, etc. The formulation may be prepared easily according to a known method using a surfactant, an excipient, a colorant, a fragrance, a preservative, a stabilizer, a buffer, a suspending agent or other commonly used adjuvants.

The pharmaceutical composition according to the present disclosure may be administered orally, rectally, topically, transdermally, intravenously, intramuscularly, intraperitoneally, subcutaneously, etc.

The administration dosage of the active ingredient will vary depending on the age, sex and body weight of a subject to be treated, the particular disease or pathological condition to be treated, the severity of the disease or pathological condition, administration route and the discretion of a diagnoser. Determination of the administration dosage based on these factors is within the level of those skilled in the art. In general, the administration dosage is 0.001-2000 mg/kg/day, more specifically 0.5-1500 mg/kg/day.

In an aspect of the present disclosure, the composition may be a health food composition. The present disclosure provides a food additive or a functional food in various forms. The composition may be prepared into fermented milk, cheese, yogurt, juice, probiotics, health food supplements and other food additives of various types.

In an exemplary embodiment, the composition may contain other ingredients that may provide a synergic effect in an amount not negatively affecting the main effect desired in the present disclosure. For example, it may further contain an additive such as a fragrance, a pigment, a sterilizer, an antioxidant, an antiseptic, a humectant, a thickener, a mineral, an emulsifier, a synthetic polymer, etc. to improve physical properties. In addition, it may further contain an auxiliary ingredient such as a water-soluble vitamin, an oil-soluble vitamin, a polypeptide, a polysaccharide, a seaweed extract, etc. These ingredients can be selected by those skilled in the art without difficulty depending on the formulation or purpose. The addition amount can be selected within the range not negatively affecting the purpose and effect of the present disclosure. For example, the addition amount may be 0.01-5 wt %, more specifically 0.01-3 wt %, based on the total weight of the composition.

The composition according to the present disclosure may be prepared into various formulations, including a solution, an emulsion, a viscous mixture, a tablet, a powder, etc., and may be administered by various methods, including simple drinking, injection, spraying, squeezing, etc.

In an aspect, the present disclosure may provide a method for anti-oxidation, which comprises applying an effective amount of the composition containing a Glycine gracilis oil to a subject. For example, the method may comprise applying the composition to the skin of the subject.

In another aspect, the present disclosure may provide a method for anti-aging, which comprises applying an effective amount of the composition containing a Glycine gracilis oil to a subject. For example, the method may comprise applying the composition to the skin of the subject.

The method for anti-aging may delay or improve aging. For example, it may delay or improve skin aging. The method may suppress or inhibit the production of collagenase or suppress the expression of collagenase. The method may improve or alleviate wrinkles.

In another aspect, the present disclosure may provide a method for skin whitening, which comprises applying an effective amount of the composition containing a Glycine gracilis oil to a subject. For example, the method may comprise applying the composition to the skin of the subject.

The method for skin whitening may exhibit a skin whitening effect and may suppress or inhibit the production of melanin or suppress the expression of melanin.

In another aspect, the present disclosure may provide a method for skin anti-inflammation, which comprises applying an effective amount of the composition containing a Glycine gracilis oil to a subject. For example, the method may comprise applying the composition to the skin of the subject.

The method for skin anti-inflammation may suppress or inhibit the production or expression of monocyte chemoattractant protein (MCP). The inflammation may comprise acne.

In another aspect, the present disclosure may provide a method for skin whitening, which comprises applying an effective amount of the composition containing a Glycine gracilis oil to a subject. For example, the method may comprise applying the composition to the skin of the subject. The method may strengthen the skin barrier and may treat, prevent or alleviate atopy or psoriasis.

In an aspect, the present disclosure may provide a Glycine gracilis oil for anti-oxidation of a subject. For example, the Glycine gracilis oil may be applied to the skin of the subject.

In an aspect, the present disclosure may provide a Glycine gracilis oil for skin anti-aging of a subject. For example, the Glycine gracilis oil may be applied to the skin of the subject.

The Glycine gracilis oil may delay or improve aging. For example, it may delay or improve skin aging. The Glycine gracilis oil may suppress or inhibit the production of collagenase or suppress the expression of collagenase. Also, it may improve or alleviate wrinkles.

In an aspect, the present disclosure may provide a Glycine gracilis oil for skin whitening of a subject. For example, the Glycine gracilis oil may be applied to the skin of the subject.

The Glycine gracilis oil may exhibit a skin whitening effect and may suppress or inhibit the production of melanin or suppress the expression of melanin.

In an aspect, the present disclosure may provide a Glycine gracilis oil for preventing, inhibiting or alleviation inflammation of a subject. For example, the Glycine gracilis oil may be applied to the skin of the subject.

The Glycine gracilis oil may suppress or inhibit the production of monocyte chemoattractant protein (MCP).

In an aspect, the present disclosure may provide a Glycine gracilis oil for skin moisturization of a subject. For example, the Glycine gracilis oil may be applied to the skin of the subject.

The Glycine gracilis oil may strengthen the skin barrier and may treat, prevent or improve atopy or psoriasis.

### Mode for Invention

Hereinafter, the present disclosure will be described in detail through examples. However, the following examples are for illustrative purposes only and the scope of the present disclosure is not limited by the examples.

### EXAMPLE

### Preparation of Glycine gracilis Oil

Glycine gracilis was washed with purified water, dried and ground finely. A Glycine gracilis oil was obtained by extracting 1 kg of the resulting Glycine gracilis powder with a hydraulic oil press machine and then filtering through filter paper. As a result, 155 g of a Glycine gracilis oil was obtained.

### Comparative Example 1

### Preparation of Soybean Oil

Soybean (Glycine max) was washed with purified water, dried and ground finely. A soybean oil was obtained by extracting 1 kg of the resulting soybean powder with a hydraulic oil press machine and then filtering through filter paper. As a result, 185 g of a soybean oil was obtained.

### Comparative Example 2

### Preparation of Glycine gracilis Extract

Glycine gracilis was washed with purified water, dried and ground finely. 100 g of the resulting Glycine gracilis powder was added to 1 L of a 70% ethanol aqueous solution, extracted by boiling in an extractor equipped with a condenser for 12 hours and then filtered through 300-mesh filter cloth. The obtained filtrate was aged at 4-15° C. for 7 days and then filtered through Whatman filter paper No. 2. The resulting filtrate was concentrated under reduced pressure using a distillation apparatus equipped with a condenser, concentrated under reduced pressure at 50° C. and then dried to obtain a Glycine gracilis extract (dry weight: 15.18 g).

### Test Example 1

### Comparison of Ingredients

The ingredients of the Glycine gracilis oil prepared in Example, the soybean oil prepared in Comparative Example 1 and the Glycine gracilis extract prepared in Comparative Example 2 were analyzed. Each of the oils and the extract was prepared into a 10,000 ppm solution by dissolving in 20% methyl chloride and 70% ethanol and the ingredients were analyzed by HPLC (Waters, model 2695, 2996 PDA detector). Kanto Chemical's Mightysil RP-18 GP 250-4.6 (5 μm) column was used as a stationary phase. The mobile phase for the Glycine gracilis oil prepared in Example and the soybean oil prepared in Comparative Example 1 is described in Table 1 and the mobile phases for the Glycine gracilis extract prepared in Comparative Example 2 is described in Table 2.

TABLE 1

| Time | A: methanol | B: acetonitrile |
|---|---|---|
| Isocratic | 20% | 80% |

TABLE 2

| | A: water | B: acetonitrile |
|---|---|---|
| Time (min) | (0.1% acetic acid) | (0.1% acetic acid) |
| 0 | 95% | 5% |
| 5 | 95% | 5% |
| 3 | 40% | 60% |
| 50 | 5% | 95% |
| 55 | 95% | 5% |

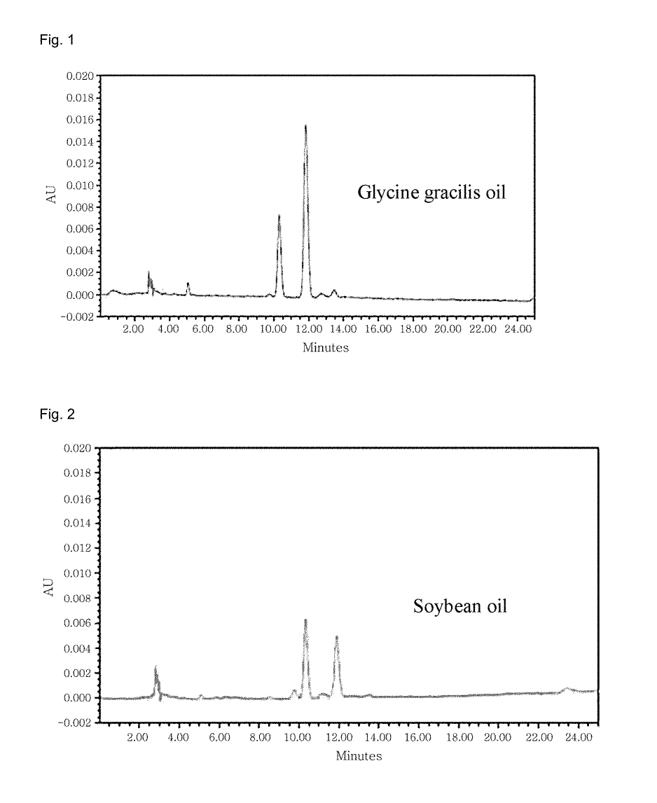

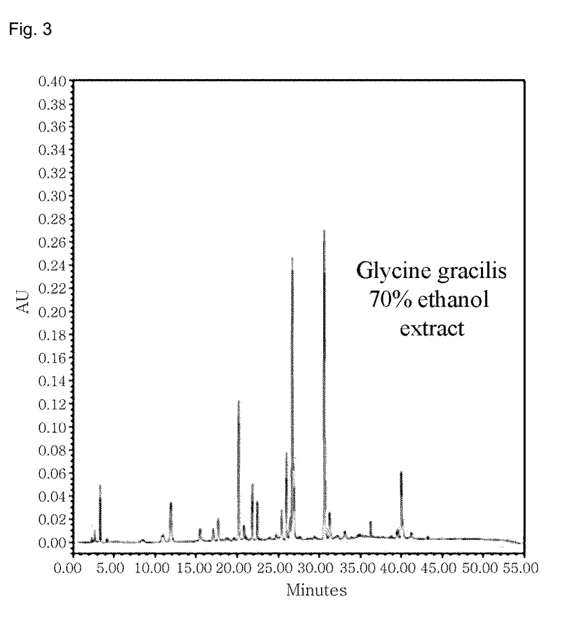

As can be seen from FIGS. 1-3 (FIG. 1: the Glycine gracilis oil prepared in Example, FIG. 2: the soybean oil prepared in Comparative Example 1; FIG. 3: the Glycine gracilis extract prepared in Comparative Example 2), the Glycine gracilis oil, which is the active ingredient of the present disclosure, shows distinct difference in peak locations and intensities from those of the soybean oil and the Glycine gracilis extract.

### Test Example 2

### Evaluation of Antioxidative Effect

The 1,1-diphenyl-2-picrylhydrazyl (DPPH) method was used to investigate the antioxidative effect of the Glycine gracilis oil prepared in Example. The DPPH method evaluates antioxidative effect by measuring and comparing the effect of suppressing DPPH oxidation based on the change in absorbance caused by the reduction of DPPH. The decrease in absorbance by the Glycine gracilis oil prepared in Example as compared to that of a control group due to suppression of DPPH oxidation was measured and the concentration at which the absorbance was decreased to 50% or below as compared to the control group was recorded.

As positive controls, the synthetic antioxidant Trolox, the soybean oil prepared in Comparative Example 1 and the Glycine gracilis extract prepared in Comparative Example 2 were used. A reaction solution was prepared by mixing 190 μL of a 100 μM DPPH solution (in ethanol) with 10 μL of each of the Glycine gracilis oil prepared in Example and the positive controls. After incubation at 37° C. for 30 minutes, absorbance was measured at 540 nm.

The analysis result is shown in Table 3, where IC₅₀ means the sample concentration when the absorbance was decreased by 50% due to the added sample.

TABLE 3

| Substances | IC₅₀ (ppm) |
|---|---|
| Glycine gracilis oil | 62 |
| Trolox | 57 |
| Soybean oil | 350 |
| Glycine gracilis extract | 71 |

As can be seen from Table 3, the Glycine gracilis oil of the present disclosure exhibits an antioxidative effect similar to that of Trolox which is a commercially available antioxidant. This effect is remarkably superior to that of the soybean oil.

### Test Example 3

### Comparison of Collagenase Expression Suppressing Effect

The effect of suppressing collagenase production of the Glycine gracilis oil prepared in Example was compared with those of tocopherol, EGCG, the soybean oil prepared in Comparative Example 1 and the Glycine gracilis extract prepared in Comparative Example 2 as positive controls. Tocopherol and EGCG are known to prevent skin aging by regenerating epidermal cells.

Human fibroblasts were added to a 96-well microtiter plate containing Dulbecco's modified Eagle's medium (DMEM) supplemented with 2.5 wt % fetal bovine serum, with 5,000 cells/well, and cultured to about 90% confluence. Then, after culturing in serum-free DMEM for 24 hours and treating with each of the Glycine gracilis oil at 100 μL/mL, 10⁻⁴ M tocopherol, 10⁻⁴ M EGCG, the soybean oil at 100 μL/mL and the Glycine gracilis extract at 100 μL/mL for 24 hours, the cell culture was harvested.

The production of collagenase in the cell culture was measured using a collagenase measuring system (Amersham Pharmacia, USA). First, the harvested cell culture was added to a 96-well plate having primary collagenase antibodies applied uniformly thereto and was incubated for 3 hours in a constant-temperature bath. 3 hours later, after adding chromophore-conjugated secondary collagen antibodies to the 96-well plate, incubation was performed for 15 minutes. 15 minutes later, a substance inducing color development was added and color development was induced at room temperature for 15 minutes. When 1 M sulfuric acid was added to stop the reaction (color development), the reaction solution exhibited a yellow color. The intensity of the yellow color varied depending on the degree of progress of the reaction.

The absorbance of the 96-well plate exhibiting yellow color was measured at 405 nm using a spectrophotometer and the degree of collagenase synthesis was calculated using Equation 1. A cell culture obtained without treating with the composition was used as a control group. That is to say, the expression level of collagenase in the group treated with the test substance was calculated relative to that of the untreated group as 100. The result is shown in Table 4.

Collagenase expression level(%)=Absorbance of group treated with test substance/Absorbance of control group×100 [Equation 1]

TABLE 4

| Substances | Collagenase expression level (%) |
|---|---|
| Untreated group | 100 |
| Tocopherol | 81 |
| EGCG | 59 |
| Glycine gracilis oil | 64 |
| Soybean oil | 87 |
| Glycine gracilis extract | 71 |

A lower collagenase expression level means a higher collagenase expression suppressing effect, which reduces wrinkles because of decreased degradation of collagen in skin.

From Table 4, it can be seen that the Glycine gracilis oil, which is the active ingredient of the present disclosure, effectively suppresses collagenase expression in vitro. Its ability of suppressing collagenase expression was even better than that of tocopherol which is known as an antioxidant. In particular, the Glycine gracilis oil, which is the active ingredient of the present disclosure, shows a higher collagenase expression suppressing effect than the soybean oil. Accordingly, it can be seen that the Glycine gracilis oil according the present disclosure can reduce the degradation of collagen in skin by effectively suppressing collagenase expression and thus exhibits excellent anti-aging and anti-wrinkling effects.

### Test Example 4

### Evaluation of Skin Whitening Effect

The melanin production suppressing effect of the Glycine gracilis oil prepared in Example was compared with those of hydroquinone, the soybean oil prepared in Comparative Example 1 and the Glycine gracilis extract prepared in Comparative Example 2 as positive controls.

Melanocytes derived from C57BL/6 mouse (Mel-Ab cells) (Dooley, T. P. et al, Skin Pharmacol, 7, pp. 188-200) were cultured in DMEM supplemented with 10% fetal bovine serum, 100 nM 12-O-tetradecanoylphorbol-13-acetate and 1 nM cholera toxin under the condition of 37° C. and 5% CO₂. The cultured Mel-Ab cells were detached with 0.25% trypsin-EDTA and cultured in a 24-well plate with a concentration of 10⁵ cells/well. During the culturing, each test substance was added for 3 consecutive days starting from day 2. The concentration of the hydroquinone, the soybean oil, the Glycine gracilis extract and the Glycine gracilis oil of Example was 10 ppm. After removing the medium, the cells were washed with phosphate-buffered saline (PBS) and lysed with 1 N sodium hydroxide. After measuring absorbance at 400 nm, the melanin production inhibition was calculated according to Equation 2. The result is shown in Table 5 (Dooley's method).

Melanin production inhibition(%)=100−(Absorbance of group treated with test substance/Absorbance of control group×100) [Equation 2]

TABLE 5

| Substances | Melanin production inhibition (%) |
|---|---|
| Untreated group | 0 |
| Hydroquinone | 57.5 |
| Glycine gracilis oil | 46.3 |
| Soybean oil | 83.9 |
| Glycine gracilis extract | 53.9 |

As seen from Table 5, the Glycine gracilis oil, which is the active ingredient of the present disclosure, exhibits better melanin production inhibition than hydroquinone which is known as a skin whitening substance. In particular, it exhibits better melanin production inhibition than the soybean oil and the Glycine gracilis extract. Accordingly, it can be seen that the Glycine gracilis oil according the present disclosure exhibits an excellent skin whitening effect by effectively suppressing melanin production in skin.

### Test Example 5

### Evaluation of Anti-Inflammatory Effect

One day before the testing, normal human skin keratinocytes (NHEKs, Lonza) were added to a 96-well plate with 5×10⁴ cells/well and cultured in a 5% CO₂ incubator at 37° C. for 24 hours. 24 hours later, the cells were washed with PBS twice and the cell culture medium was replaced with a serum-free keratinocyte basement medium (KBM).

Then, the cells were incubated for 30 minutes with each of hydrocortisone, the soybean oil prepared in Comparative Example 1 and the Glycine gracilis extract prepared in Comparative Example 2, as positive controls, and the Glycine gracilis oil prepared in Example at the concentrations described in Table 4 and then each well was treated with 10 μM retinoic acid. The retinoic acid is known to promote the secretion of the inflammatory cytokine MCP (monocyte chemoattractant protein-1) as a skin irritant. After incubation in a 5% CO₂ incubator at 37° C. for 24 hours, MCP-1 ELISA was conducted. The result is shown in Table 6. For the ELISA, the method provided by BD Science was used.

TABLE 6

| | MCP-1 |
|---|---|
| | secretion |
| Substances | (pg/mL) |
| Untreated group (negative control) | 527.3 |
| Retinoic acid (10 μM) | 804.5 |
| Hydrocortisone (100 nM) + retinoic acid (10 μM) | 448.3 |
| Soybean oil (100 ppm) + retinoic acid (10 μM) | 795.3 |
| Glycine gracilis extract (100 ppm) + retinoic acid (10 μM) | 683.5 |
| Glycine gracilis oil (100 ppm) + retinoic acid (10 μM) | 535.8 |

As can be seen from Table 6, the Glycine gracilis oil, which is the active ingredient of the present disclosure, remarkably decreases the secretion of MCP-1 which has been increased by retinoic acid. This result was comparable to that of hydrocortisone which is commercially available as an anti-inflammatory substance and was remarkably better than those of the Glycine gracilis extract and the soybean oil. Accordingly, it can be seen that the Glycine gracilis oil, which is the active ingredient of the present disclosure, has a superior anti-inflammatory effect.

### Test Example 6

### Evaluation of Skin Moisturizing Effect

The skin barrier function and skin moisturizing effect of the Glycine gracilis oil prepared in Example was investigated by measuring absorbance.

Primarily cultured human keratinocytes were placed in a culture flask and allowed to adhere to the flask bottom. Then, after adding each of the soybean oil prepared in Comparative Example 1 at 50 mg/mL and 100 mg/mL, the Glycine gracilis extract prepared in Comparative Example 2 at 50 mg/mL and 100 mg/mL and the Glycine gracilis oil prepared in Example at 50 mg/mL and 100 mg/mL, the cells were cultured for 5 days until the cells reached a confluence of about 80-90%. The cells were harvested and washed with phosphate-buffered saline (PBS). After adding 1 mL of a 10 mM Tris-HCI buffer (pH 7.4) containing 2% sodium dodecyl sulfate (SDS) and 20 mM dithiothreitol (DTT), the cells were sonicated for 3 minutes and then boiled for 10 minutes. After centrifuging at 1200 rpm for 30 minutes, the separated pellets were resuspended in 1 mL of PBS and absorbance was measured at 340 nm.

Separately from this, a portion of the solution after the sonication was sampled and the protein content thereof was measured for use as a reference in evaluation of cellular differentiation. A group treated with low-concentration calcium (0.03 mM) and a group treated with high-concentration calcium (1.2 mM) were used as a negative control group and a positive control group, respectively. The result of tests performed by adding the test substances to low-concentration calcium is shown in Table 7.

TABLE 7

| | | | Differentiation of |
|---|---|---|---|
| Substances | Concentration | | keratinocytes (%) |
| Control | Low Ca²⁺ | (0.03 mM) | 100 |
| | High Ca²⁺ | (1.2 mM) | 195 |
| Glycine gracilis extract | 100 | mg/mL | 171 |
| | 50 | mg/mL | 135 |
| Glycine gracilis oil | 100 | mg/mL | 182 |
| | 50 | mg/mL | 146 |
| Soybean oil | 100 | mg/mL | 138 |
| | 50 | mg/mL | 116 |

From Table 7, it can be seen that the Glycine gracilis oil according to the present disclosure promotes differentiation of keratinocytes when the effect of promoting cellular differentiation was compared by measuring the amount of cornified envelopes (CE) produced during the differentiation of keratinocytes. In particular, the effect of promoting the differentiation of keratinocytes was remarkably higher than that of the Glycine gracilis extract or the soybean oil. Accordingly, it can be seen that the Glycine gracilis oil according the present disclosure can strengthen the skin barrier function and enhances skin moisturization.

### Test Example 7

### Evaluation of Skin Irritation

Kojic acid as a known skin whitening substance and the Glycine gracilis oil, which is used as the active ingredient in the present disclosure, were tested comparatively for irritation by 15 panels who are sensitive to skin irritation such as pricking, flushing, etc.

The panels were asked to apply 0.5 mL of kojic acid (purchased from YM Chemical) or the Glycine gracilis oil prepared in Example randomly on each cheek and score between 0 and 3.0 points with 0.1 point intervals. The result is shown in Table 8.

TABLE 8

| | Kojic acid | Glycine gracilis oil |
|---|---|---|
| Pricking | 0.81 | 0.19 |
| Flushing | 0.43 | 0.17 |
| Mean | 0.62 | 0.18 |

<Scoring Standard>

0-0.4: No irritation.

0.5-1.0: Slight irritation.

1.1-2.0: Moderate irritation.

2.1-3.0: Severe irritation.

As can be seen from Table 8, kojic acid showed noticeable skin irritation such as pricking and flushing. In contrast, the Glycine gracilis oil, which is the active ingredient of the present disclosure, greatly decreased the skin irritation.

The composition of the present disclosure can be prepared into various formulations as described below, although not being limited thereto.

### Preparation Example 1

### Tablet

A granule prepared by mixing 100 mg of Glycine gracilis oil, 100 mg of glucose, 50 mg of red ginseng extract, 96 mg of starch and 4 mg of magnesium stearate and then adding 40 mg of 30% ethanol was dried at 60° C. and prepared into a tablet using a tablet making machine.

### Preparation Example 2

### Granule

A granule prepared by mixing 100 mg of Glycine gracilis oil, 100 mg of glucose, 50 mg of red ginseng extract and 600 mg of starch and then adding 100 mg of 30% ethanol was dried at 60° C. and filled in a pouch. The final weight was 1 g.

### Preparation Example 4

### Drink

100 mg of Glycine gracilis oil, 10 g of glucose, 50 mg of red ginseng extract, 2 g of citric acid and 187.8 g of purified water were mixed and filled in a bottle. The final volume was 200 mL.

### Preparation Example 5

### Health Food

TABLE 9

| Ingredients | Contents | |
|---|---|---|
| Glycine gracilis oil | 1000 | mg |

Vitamin mixture

| | | |
|---|---|---|
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

Although the above-described composition of vitamin and mineral mixtures is given as a specific example relatively adequate for a health food, it may also be altered as desired. After mixing the above ingredients and preparing into a granule, a health food was prepared according to a commonly employed method.

### Preparation Example 6

### Health Drink

TABLE 10

| Ingredients | Contents | |
|---|---|---|
| Glycine gracilis oil | 1000 | mg |
| Citric acid | 1000 | mg |
| Oligosaccharide | 100 | g |
| Plum concentrate | 2 | g |
| Taurine | 1 | g |
| Purified water | Balance | |
| Total volume | 900 | mL |

As described in Table 10, the above ingredients were mixed and purified water was added to make the total volume 900 mL. After heating at 85° C. for about 1 hour under stirring, the resulting solution was filtered and filled in a sterilized 2-L container. After sealing and sterilizing, the solution was stored in a refrigerator until used to prepare a health drink composition of the present disclosure.

### Formulation Example 1

### Lotion

TABLE 11

| Ingredients | Contents (wt %) |
|---|---|
| Glycine gracilis oil | 3.00 |
| L-Ascorbic acid 2-phosphate magnesium salt | 1.00 |
| Water-soluble collagen (1% aqueous solution) | 1.00 |
| Sodium citrate | 0.10 |
| Citric acid | 0.05 |
| Celandine extract | 0.20 |
| 1,3-Butylene glycol | 3.00 |
| Purified water | To 100 |

### Formulation Example 2

### Cream

TABLE 12

| Ingredients | Contents (wt %) |
|---|---|
| Glycine gracilis oil | 1.00 |
| Polyethylene glycol monostearate | 2.00 |
| Self-emulsifying glyceryl monostearate | 5.00 |
| Cetyl alcohol | 4.00 |
| Squalane | 6.00 |
| Glyceryl tri-2-ethylhexanoate | 6.00 |
| Sphingoglycolipid | 1.00 |
| 1,3-Butylene glycol | 7.00 |
| Purified water | To 100 |

### Formulation Example 3

### Pack

TABLE 13

| Ingredients | Contents (wt %) |
|---|---|
| Glycine gracilis oil | 5.00 |
| Polyvinyl alcohol | 13.00 |
| L-Ascorbic acid 2-phosphate magnesium salt | 1.00 |
| Lauroyl hydroxyproline | 1.00 |
| Water-soluble collagen (1% aqueous solution) | 2.00 |
| 1,3-Butylene glycol | 3.00 |
| Ethanol | 5.00 |
| Purified water | To 100 |

### Formulation Example 4

### Beauty Solution

TABLE 14

| Ingredients | Contents (wt %) |
|---|---|
| Glycine gracilis oil | 2.00 |
| Hydroxyethyl cellulose (2% aqueous solution) | 12.00 |
| Xanthan gum (2% aqueous solution) | 2.00 |
| 1,3-Butylene glycol | 6.00 |
| Thick glycerin | 4.00 |
| Sodium hyaluronate (1% aqueous solution) | 5.00 |
| Purified water | To 100 |

### Formulation Example 5

### Ointment

TABLE 15

| Ingredients | Contents (wt %) |
|---|---|
| Glycine gracilis oil | 0.1 |
| Glycerin | 8.0 |
| Butylene glycol | 4.0 |
| Liquid paraffin | 15.0 |
| β-Glucan | 7.0 |
| Carbomer | 0.1 |
| Caprylic/capric triglyceride | 3.0 |
| Squalane | 1.0 |
| Cetearyl glucoside | 1.5 |
| Sorbitan stearate | 0.4 |
| Cetearyl alcohol | 1.0 |
| Beeswax | 4.0 |
| Antiseptic, pigment and flavor | Adequate |
| Purified water | Balance |

While the exemplary embodiments have been shown and described, it will be understood by those skilled in the art that various changes in form and details may be made thereto without departing from the spirit and scope of this disclosure as defined by the appended claims.

1. A method for improving skin condition of a subject, comprising:
administering an effective amount of a composition comprising a Glycine gracilis oil as an active ingredient to a subject in need thereof.

2. The method according to claim 1, wherein the improving skin condition of the subject comprises anti-aging.

3. The method according to claim 2, wherein the method delays or improves aging.

4. The method according to claim 2, wherein the method suppresses or inhibits the production of collagenase or suppresses the expression of collagenase.

5. The method according to claim 2, wherein the method improves or alleviates wrinkles.

6. The method according to claim 1, wherein the improving skin condition of the subject comprises skin whitening.,

7. The method according to claim 6, wherein the method suppresses or inhibits the production of melanin or suppresses the expression of melanin.

8. The method according to claim 1, wherein the improving skin condition of the subject comprises anti-inflammation.

9. The method according to claim 8, wherein the inflammation is acne.

10. The method according to claim 8, wherein the method suppresses or inhibits the production or expression of monocyte chemoattractant protein (MCP).

11. The method according to claim 1, wherein the improving skin condition of the subject comprises skin moisturization.

12. The method according to claim 11, wherein the method treats, prevents or improves atopy or psoriasis.

13. The method according to claim 11, wherein the method strengthens skin barrier.

14. The method according to claim 1, wherein the composition contains 0.001-10 wt % of a Glycine gracilis oil.

15. The method according to claim 1, wherein the composition is selected from a group consisting of a cosmetic composition, a composition for external application to skin, a pharmaceutical composition, and a health food composition.

16-18. (canceled)

19. The method according to claim 1, wherein the improving skin condition of the subject comprises anti-oxidation.

20. A method for anti-oxidation of a subject, comprising:
administering an effective amount of a composition comprising a Glycine gracilis oil as an active ingredient to a subject in need thereof.

21. The method according to claim 20, wherein the composition contains 0.001-10 wt % of a Glycine gracilis oil.

22. The method according to claim 20, wherein the composition is selected from a group consisting of a cosmetic composition, a composition for external application to skin, a pharmaceutical composition, and a health food composition.
